(19) United States
(12) Patent Application Publication
(10) Pub. No.: US 2019/0151222 A1
(43) Pub. Date: May 23, 2019

(54) ONE PART ARTIFICIAL NAIL SCULPTING COMPOSITIONS AND METHODS WITHOUT REACTIVE OLIGOMERS

(71) Applicant: NAIL ALLIANCE, LLC, Gladstone, MO (US)

(72) Inventors: Sunil SIRDESAI, Irvine, CA (US); Danny Lee HAILE, La Habra, CA (US)

(21) Appl. No.: 16/195,688

(22) Filed: Nov. 19, 2018

Related U.S. Application Data
(60) Provisional application No. 62/588,165, filed on Nov. 17, 2017.

(51) Int. Cl. A61K 8/81; A61Q 3/02; A61K 8/92; A45D 31/00

(52) U.S. Cl. CPC A61K 8/8147; A61Q 3/02; A61K 8/922; A45D 2200/205; A45D 31/00; A61K 2800/81; A61K 2800/30; A61K 8/8158

(57) ABSTRACT

Photopolymerizable compositions for forming a cosmetic nail coating or artificial nail and methods of using such compositions. The compositions include (a) from about 15 wt % to about 45 wt % of at least one rheology modifier; (b) from about 25 wt % to about 75 wt % of one or more (meth)acrylate monomers; (c) one or more photoinitiators; and optionally (d) from about 15 wt % to about 45 wt % of one or more binding agents. The compositions may be a liquid or a gel capable of forming an artificial nail or cosmetic nail coating in one application and without being mixed with any additional components. The composition may include only inert polymers.

### CROSS-REFERENCE TO RELATED APPLICATIONS

This application claims priority to U.S. provisional application No. 62/588,165 filed Nov. 17, 2017, which is hereby incorporated by reference in its entirety.

### FIELD

The present disclosure is directed generally to artificial nail compositions. More specifically, the present disclosure is directed to one part artificial nail sculpting compositions without reactive oligomers.

### BACKGROUND

Acrylic nails are used to artificially enhance the appearance of natural fingernails. Various forms of this product are used throughout the world, including press-on nails, two part (monomeric liquid and polymeric powder) systems, and UV gel systems.

Press-on acrylic nails, introduced to the manicure industry in the early 1970s, are nail-shaped pieces of polymer that are glued onto natural nails. Subsequent developments afforded more natural-looking nail enhancements which bond to the real nail by an acrylic-based resin. Such a resin is created by mixing a liquid and powder together to form a thick paste. A nail technician applies the paste over the natural nail and allows it to harden to form a durable nail coating finish that is filed into the desired shape.

Although press-on acrylic nails are used today, they have been largely replaced by sculpted acrylic nails. Sculpted acrylic nails may be formed by several different methods, but the most popular and widely-used method comprises an application of a two-part acrylic formulation by a nail technician. The nail technician typically wets a brush with a monomer liquid, dips the wetted brush into a polymeric powder to form a wet ball of dough, places the wetted ball of the blend of monomer liquid and polymer powder onto a nail and an acrylic nail form adjacent to a nail, and shapes the artificial nail.

The acrylic nail form is a substrate used for construction of acrylic nails beyond the existing free edge of the nail. Elongation requires affixing a substrate means to the exposed edge of the nail, which means has the general contour of the natural nail and extends therefrom to the desired length and along the plane of the natural nail. The substrate is typically of a material to which the polymerized mixture does not adhere, so that the form may be removed following construction of the artificial nail.

The powder portion of the two-part acrylic formulation generally comprises a solid polymer, such as poly (methyl methacrylate), poly (ethyl methacrylate), copolymers of poly (methyl methacrylate) and poly (ethyl methacrylate), and a catalyst, such as benzoyl peroxide.

The two-part acrylic formulation also comprises a liquid, generally referred to as “monomer liquid,” which comprises an (meth)acrylic monomer, such as methyl methacrylate or ethyl methacrylate, crosslinkers, additives, decomposition promoters such as amines, and optionally other ingredients needed for forming a mixture with the solid polymer to prepare an artificial nail.

One of the biggest disadvantages of the monomer liquid used in the traditional system is the offensive odor of the ethyl methacrylate. This odor may be unappetizing to most clients and its long-term exposure has been hypothesized to be dangerous to the nail technicians.

Odorless systems comprising hydroxyethyl methacrylate, hydroxypropyl methacrylate and other methacrylates have been developed, but they generally have the disadvantage of poor cure (inhibited layer or tacky surface) and finished aesthetics.

Another method of forming artificial nails is by the use of a UV-curable system. In one prior art method of the UV-curable system method, a brush dipped into the formulated monomer liquid is placed into the formulated polymer powder, applied to the client's finger nail, and after the artificial nails are formed, the artificial nails are cured under a UV lamp for several minutes.

A UV-cured acrylic system has the same odor disadvantages as compared to traditional acrylic systems. However, many nail technicians find that UV-curable systems are difficult to work with and are presently typically only used to create overlays, and not free-standing nail extensions. Further, another disadvantage of typical UV-cured two-part powder systems are brittleness of the formed nail.

Accordingly, one part artificial nail compositions having one or more advantageous characteristics and/or that overcome problems associated with UV cured artificial nail systems, including two-part powder systems, are desired.

### SUMMARY

The present disclosure provides photopolymerizable compositions for forming an artificial nail or a cosmetic coating for nails comprising: (a) from about 15 wt % to about 45 wt % of at least one rheology modifier (b) from about 25 wt % to about 75 wt % of one or more (meth)acrylate monomers; (c) one or more photoinitiators; and optionally (d) one or more binding agents; and optionally (e) one or more crosslinkers; wherein all wt % are with respect to the photopolymerizable composition. This composition exhibits high viscosity that resembles the viscosity of a typical traditional two part system about one minute after the monomer liquid and the polymer have been mixed. This composition can be shaped or sculpted readily by a nail technician to form nail coatings without any additional use of monomer liquid, solvent, oil, or mixtures thereof, followed by an exposure to UV light to photopolymerize the composition to create a hard, durable nail coating. Optionally, the presently disclosed compositions may be contacted with a patting liquid to aid in the shaping of the composition to form a nail coating or artificial nail of a desired shape. In some instances, a brush being used for shaping can be wetted with a patting liquid such as a monomer liquid, solvent, oil, slip agent, or mixtures thereof. Additional components such as colorants and antioxidants may be included in the composition of the present disclosure.

According to at least one aspect of the present disclosure, the presently disclosed compositions do not include reactive oligomers that may be found in other sculpting compositions. In this aspect, the presently disclosed compositions include only inert polymers rather than the reactive oligomers and reactive polymers relied upon in other sculpting compositions. Reactive oligomers and polymers, besides providing body to the formulation, chemically react to form a network. In contrast, inert oligomers or polymers do not participate in forming a network.

The presently disclosed composition solves one or more problems associated with the formation of a nail coating or nail extension using either procedures relying on gel nail formulations or acrylic nail formulations. According to at least one aspect of the present disclosure, the presently disclosed compositions may exhibit one or more of the following non-advantages: (1) a high viscosity composition which is easy to apply either in the neat form or patted with a liquid; (2) a composition that remains firm and does not run during application to the nail; (3) a one part formulation that does not need to be mixed reducing the time required to serve the client; (4) eliminates problems preparing the correct mixing ratios necessary to produce a workable composition; (5) render moot the lack of heterogeneity inherent to formulations associated with two-part formulations; (6) exhibit a lack of odor characteristic of one-part formulations; (7) exhibit a lower exotherm profile compared to the existing builder (sculpting) gel formulations mitigating unpleasantness or pain to the client; (8) exhibit the ability to apply the composition in thicker layers when creating nail extensions; (9) exhibits excellent adhesion; (10) exhibits abrasion resistance. According to at least one aspect of the present disclosure, the mechanical strength of the cured nail coating produced by the presently disclosed compositions may be as good as that obtained by liquid/powder systems but is 23% lighter. Additionally, the presently disclosed compositions are characterized by self-leveling properties that are superior to traditional acrylic nail systems, exhibit no significant shrinkage, exhibit suitable stability for commercial viability as a one part composition, results in a reduction of the yellowing of the cosmetic nail coating, and provides a very long open time to sculpt the nail.

According to at least one aspect of the present disclosure, the presently disclosed compositions may include from about 15 wt % to about 45 wt % of one or more binding agents. In at least some instances, the one or more binding agents may be selected from the group consisting of polyisobutene, ozokerite wax, ceresin, and a copolymer of acrylamide and sodium acryloyldimethyltaurate. In at least some instances, the binder may comprise a mixture of acrylamide, sodium acryloyldimethyl taurate copolymer, isohexadecane, and polysorbate 80. In other instances, the binder may comprise a mixture of polyacrylamide, C13-14 isoparaffin, and laureth-7. In some cases, the binder may be a copolymer of acrylamide and sodium acryloyldimethyltaurate. According to at least one aspect of the present disclosure, the one or more binding agents is not a wax. In some instances, the presently disclosed compositions exclude a wax. In some instances, the binder may exclude a wax.

According to at least one aspect of the present disclosure, the at least one rheology modifier is selected in an amount such that the viscosity of the composition is greater than 400,000 centipoise (cps) at 25° C. as measured by a cone and plate rheometer using 40 mm 1° steel cone with a truncation gap of 31 microns at a 10 s⁻¹ shear rate on a 500 mg sample. In some instances, the at least one rheology modifier is selected in an amount such that the viscosity of the composition is from about 600,000 cps to about 1,200,000 cps at 25° C. as measured by a cone and plate rheometer using 40 mm 1° steel cone with a truncation gap of 31 microns at a 10 s⁻¹ shear rate on a 500 mg sample. In at least some instances, the at least one rheology modifier is selected in an amount such that the viscosity of the composition is from about 1,100,000 cps to about 3,250,000 cps at 25° C. as measured by a Brookfield Viscometer using spindle 96 at 0.3 rpm at the 1 minute mark. In some cases, the at least one rheology modifier is selected in an amount such that the viscosity of the composition is from about 2,500,000 cps to about 3,150,000 cps at 25° C. as measured by a Brookfield Viscometer using spindle 96 at 0.3 rpm at the 1 minute mark.

According to at least one aspect of the present disclosure, the rheology modifier may be selected from the group consisting of methacrylate polymers, methacrylate copolymers, styrene polymers, styrene copolymers, polyethylene, polyesters, polysulfides, polycarbonates, silicones, carboxylic esters, and any combination thereof. The presently disclosed compositions may include from about 15 wt % to about 45 wt % of the at least one rheology modifier. The presently disclosed compositions may also include from about 20 wt % to about 35 wt % of the at least one rheology modifier. In some instances, the presently disclosed compositions may include from about at least one rheology modifier may comprise from about 15 wt % to about 20 wt %, or from about 20 wt % to about 30 wt %, or from about 25 wt % to about 35 wt %, or from about 20 wt % to about 25 wt %, or from about 35 wt % to about 45 wt %, or from about 15 wt % to about 35 wt %, or from about 25 wt % to about 45 wt % of the at least one rheology modifier.

In at least some instances, the rheology modifier may comprise from about 15 wt % to about 45 wt % of one or more polymers comprised of the group consisting of poly(C₁₋₁₂alkyl(meth)acrylate) polymer, poly(C₁₋₁₂alkyl(meth)acrylate) copolymer, styrene polymer, styrene copolymer, and mixtures thereof. In at least some instances, the rheology modifier may be a polymer in the form of a powder and may resemble the polymer powders typically used in the acrylic nail industry. In some cases, the rheology modifier of the present disclosure can comprise one or more poly(alkyl (meth)acrylates, styrenic-type monomers (such as styrenic (meth)acrylates), or a mixture thereof, wherein the alkyl groups comprise one to twelve carbon atoms.

According to at least one aspect of the present disclosure, the rheology modifier may comprise a mean particle size of from about 1 micrometers (μm) to about 100 μm. In some instances, the rheology modifier may comprise a mean particle size of less than about 100 μm. In other cases, the rheology modifier may comprises a mean particle size of less than about 50 μm, or less than about 10 μm, or less than about 5 μm. In some instances, the polymer powder consists of fine microspheres with a particle size of from about 1 μm to about 100 μm, or less than 50 μm, or less than 10 μm, or less than 5 μm.

According to at least one aspect of the present disclosure, the rheology modifier may be a polymer that is insoluble in the composition. In some instances, the rheology modifier may be a polymer that forms a suspension of the polymer in the composition.

The presently disclosed photopolymerizable compositions for forming a cosmetic coating for nails may also comprise about 25 wt % to about 75 wt % of one or more (meth)acrylate monomers. The (meth)acrylate monomer used in the presently disclosed compositions may be any acrylate monomer or methacrylate monomer that is used in nail art formulations in which the curing is performed by UV light. The (meth)acrylate monomer may have a formula CH₂═C(R)—COOR′, wherein R is H, Me, or a mixture thereof, and R′ is an organic group. According to at least one aspect of the present disclosure, the at least one of the one or more (meth)acrylate monomers may comprise a hydroxyl-containing (meth)acrylate monomer.

The photopolymerizable composition of the present disclosure for forming a cosmetic coating for nails also comprises one or more photoinitiators. The photoinitiator may be selected so that it is activated by photons of the wavelength associated with UV light of the UV lamp. Preferably, the photoinitiator should be active at the wavelength of UV light of UV lamps commonly found in nail salons. Such photoinitiators may be selected from benzyl ketones, monomeric hydroxyl ketones, polymeric hydroxyl ketones, α-amino ketones, acyl phosphine oxides, metallocenes, benzophenone, and benzophenone derivatives.

The photopolymerizable compositions of the present disclosure for forming a cosmetic coating for nails may also optionally comprise one or more crosslinkers. Such crosslinkers include acrylates, methacrylates, alkoxylated crosslinker, with the formula (CH₂═CMe-C(O)—O-(AO)ₓ—CH₂—)₃C—R; wherein R is a C₁ to C₆ group with an ‘ene’ moiety; AO is a small alkoxy group; and wherein for each (CH₂═CMe-C(O)—O-(AO)ₓ—CH₂—) group x is independently 0, 1, 2, or 3.

The photopolymerizable compositions of the present disclosure may further comprise a small amount of pigment. Advantages of using a pigment may include: providing a tint or a color to the formed cosmetic nail coating; using a pigment is to give a clear or colorless or natural appearance of the cosmetic nail coating; and providing a natural appearance to the photopolymerizable composition.

The viscosity of the photopolymerizable compositions of the present disclosure is above 400,000 centipoise at 25° C. as determined by a cone and plate rheometer using 40 mm 1° steel cone with the truncation gap of 31 microns operating at 2 to 10 s⁻¹ shear rates on a 500 mg sample. In some instances, the viscosity of the presently disclosed composition is from about 600,000 cps to about 1,200,000 cps at 25° C. as measured by a cone and plate rheometer using 40 mm 1° steel cone with a truncation gap of 31 microns at a 10 s⁻¹ shear rate on a 500 mg sample. In other instances, the viscosity of the presently disclosed composition is from about 1,100,000 cps to about 3,250,000 cps at 25° C. as measured by a Brookfield Viscometer using spindle 96 at 0.3 rpm at the 1 minute mark. In still other cases, the viscosity of the presently disclosed composition is from about 2,500,000 cps to about 3,150,000 cps at 25° C. as measured by a Brookfield Viscometer using spindle 96 at 0.3 rpm at the 1 minute mark.

The photopolymerizable compositions of the present disclosure may be prepared by any means used to add and blend high viscosity compositions. Addition of the components, blending of the components and filling appropriate containers may be done at elevated temperatures.

The container in which the photopolymerizable compositions of the present disclosure is sold may be any container capable of handling high viscosity compositions and of providing protection from UV light. Examples of suitable containers include jars or pots, a tube container, and a syringe container. Each of these exemplified containers has an orifice which is designed to deliver a bead of the photopolymerizable composition. The orifice is designed so that the technician is able to deliver a uniform bead to the nail of the client.

Another aspect of the present disclosure are methods of using the presently disclosed photopolymerizable compositions to form a cosmetic coating for a nail or an artificial nail. In particular, the present disclosure provides a method of forming a cosmetic nail or an artificial nail, the method comprising: (a) contacting the presently disclosed compositions with a patting liquid selected from the group consisting of an acrylic nail monomer, solvent, oil, slip agent, photoinitiator, and any mixture thereof, to form a blend; (b) placing the blend onto a nail; and (c) exposing the blend to UV light. The present disclosure also provides a method of forming a cosmetic nail or an artificial nail, the method comprising (a) placing the presently disclosed composition onto a nail; (b) contacting the composition with a patting liquid selected from the group consisting of an acrylic nail monomer, solvent, oil, photoinitiator, and any mixture thereof; and (c) exposing the mixture to UV light.

The present disclosure also provides a method for forming a cosmetic nail coating or an artificial nail, the method comprising: applying a composition onto a nail in one application step, the composition comprising: (a) from about 15 wt % to about 45 wt % of at least one rheology modifier; (b) from about 25 wt % to about 75 wt % of one or more (meth)acrylate monomers; and (c) one or more photoinitiators; and exposing the composition to UV light; wherein the composition is in the form of a liquid or gel and is not mixed with any additional components prior to the applying of the composition onto the nail.

After the presently disclosed composition is shaped, the composition, along with finger and the hand of the client, may be exposed to UV light to cure the composition.

According to at least one aspect of the present disclosure, the rheology modifier is selected in an amount such that a 0.5 gram sample of the composition exhibits a temperature increase of less than about 20° C. during the exposure to UV light. The presently disclosed compositions may be stable in a dark container at 49° C. for four months or at 65° C. for 2 weeks.

The presently disclosed compositions may be a liquid or a gel capable of forming an artificial nail or cosmetic nail coating in one application and without being mixed with any additional components.

### DETAILED DESCRIPTION

For illustrative purposes, the principles of the present disclosure are described by referencing various exemplary embodiments thereof. Although certain embodiments of the present disclosure are specifically described herein, one of ordinary skill in the art will readily recognize that the same principles are equally applicable to, and can be employed in other compositions and methods. Before explaining the disclosed embodiments of the present disclosure in detail, it is to be understood that the present disclosure is not limited in its application to the details of any particular embodiment shown. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

As used herein and in the appended claims, the singular forms “a”, “an”, and “the” include plural references unless the context clearly dictates otherwise. The singular form of any class of the ingredients or components refer not only to one chemical species within that class, but also to a mixture of those chemical species; for example, the term “photoinitiator” in the singular form, may refer to a mixture of compounds each of which is also a photoinitiator. The terms “a” (or “an”), “one or more” and “at least one” can be used interchangeably herein. It is also to be noted that the terms “comprising,” “including,” and “having” can be used interchangeably.

The abbreviations and symbols as used herein, unless indicated otherwise, take their ordinary meaning. The abbreviation or symbol “cp” means centipoise, or millipascal-seconds. The abbreviation or symbol “μm” means micron or micrometer. The term “about” when referring to a number means ±3%. For example, the phrase “about 50 wt %” refers to a number between and including 48.500 wt % and 51.500 wt %.

The term “wt %” means percent by weight. The phrase “wherein all wt % are with respect to the photopolymerizable composition” means that the recited percent by weight values for each ingredient is compared to the entire photopolymerizable composition. The entire photopolymerizable composition includes not only ingredients which wt % are listed, but also listed ingredients for which wt % are omitted, and also ingredients that may not be recited.

The term “client” refers to a person whose nails are being treated. The phrase “nail technician” or “technician” is a worker skilled or licensed in the art of providing nail extensions, artificial nails, acrylic nails, gel nails, and other manicure services for clients. Alternative names for a nail technician may include a manicurist, or a cosmetologist. Such a person may work for pay at a nail salon, or may be a manicure aficionado.

Under one embodiment of the present invention, the client and the nail technician are two different individuals. Although the present disclosure describes the nail technician and the client as two separate individuals, it is understood that the claimed compositions and methods are also suitable for use by a single person who is both a nail technician and a client. Under another embodiment of the present disclosure, the client and the nail technician are the same person.

The term “nail,” refers to either a fingernail or a toenail. The term “nail” also refers to a human nail, as well as to any toughened keratin at the end of a digit of a nonhuman animal. The phrase “cosmetic nail coating” refers to the hardened, fully cured substance covering a part or all of the nail, and any portions of this substance that extends or is built beyond the free edge of the nail. The term “cosmetic nail coating” may also include an “artificial nail” that may be formed either on the nail of a human or nonhuman animal or that may be formed in a mold or other device in order to form an artificial nail suitable to being placed on, or applied to, a nail of a human or nonhuman animal.

The term “acrylic” in the phrase “acrylic nail” refers to a hardened polymerized composition used in manicure arts, which may be composed of any of several types of poly((meth)acrylates), or copolymers of various (meth)acrylate monomers, oligomers or copolymers of various (meth)acrylate monomers with any of several non-(meth)acrylic monomers.

When referring to a composition, the definition of the term “acrylate” as referred to in the monomeric form, includes an ester, a salt, or a conjugate base of acrylic acid, with the formula CH₂═CH—COO⁻. The definition of the term “acrylate” referred to in the polymeric or oligomeric form includes the repeating unit of an ester, a salt, or a conjugate base of acrylic acid, with the formula —[CH₂—CH(COO⁻)]—.

The definition of the term “methacrylate” as referred to in the monomeric form includes an ester, a salt, or a conjugate base of methacrylic acid, with the formula CH₂═C(CH₃)—COO⁻.

The term “(meth)acrylate” means acrylate, methacrylate, or a mixture thereof. When referring to a compound, “(meth)acrylate” means an ester, a salt, or a conjugate base of an acrylic acid, with the formula CH₂═C(R)—COO⁻, wherein R is H, Me, or a mixture thereof. By extension, a monomer or polymer name containing as a part of its term the string “(meth)acrylate” should be interpreted as referring to the same monomer or polymer, that is an acrylate, methacrylate, or a mixture thereof. For example, the term “poly(C₁₋₁₂alkyl (meth)acrylate)” means “any of poly(C₁₋₁₂alkyl acrylate), poly(C₁₋₁₂alkyl methacrylate), and a mixture of poly(C₁₋₁₂alkyl acrylate) and poly(C₁₋₁₂alkyl methacrylate).”

The term “rheology modifier” refers to any chemical compound suitable for use in a composition for forming a cosmetic coating for nails or for forming an artificial nail that may be used in the presently disclosed compositions to modify the viscosity of the composition. In at least some instances, the rheology modifier is added to the presently disclosed compositions to achieve a particular desired viscosity of the composition suitable for forming a cosmetic nail coating or an artificial nail. In at least some instances, the particular rheology modifier, or mixture of rheology of modifiers, may be selected, and in an amount, to produce a particular desired viscosity dependent on the amounts and chemical composition of the other components used in the composition.

The term “binding agent” refers to any chemical compound suitable for use in a composition for forming a cosmetic coating for nails, or for forming an artificial nail, that may be used in the presently disclosed compositions to hold all of the components of the formulation in a viscous state (semi-solid state) prior to curing and that upon curing are substantially trapped within the crosslinks formed by the monomers rather than chemically reacting to form an integral part of cross-links. In other words, the binding agents of the presently disclosed compositions are inert and therefore are trapped in the crosslinks of the composition upon curing. This is in contrast to artificial nail sculpting compositions or cosmetic nail coating compositions that include reactive oligomers which are responsible for chemically reacting to form crosslinks.

The present disclosure relates to photopolymerizable compositions for forming a cosmetic coating for nails or an artificial nail comprising: (a) from about 15 wt % to about 45 wt % of at least one rheology modifier (b) from about 25 wt % to about 75 wt % of one or more (meth)acrylate monomers; (c) one or more photoinitiators; and optionally (d) one or more binding agents; and optionally (e) one or more crosslinkers; wherein all wt % are with respect to the photopolymerizable composition. The presently disclosed compositions may exhibit high viscosity that resembles the viscosity of a typical traditional two part system about one minute after the monomer liquid and the polymer have been mixed. In at least some instances, the presently disclosed compositions can be shaped readily by a nail technician to form nail coatings without any additional use of monomers liquid, followed by an exposure to UV light to photopolymerize the compositions to create hard, durable nail coatings.

The presently disclosed compositions may comprise at least one rheology modifier, one or more monomers, one or more photoinitiators, and optionally one or more binding agents. The presently disclosed compositions may also optionally include one or more crosslinkers. Each or any of these four ingredients may be a single compound, or each or any of these ingredients may be a mixture of compounds that fall within the definition of such ingredient. Additional ingredients such as a colorant, a special effects pigment or an antioxidant may be included in the composition of the present invention.

The compositions of the present disclosure may solve one or more problems associated with the formation of a nail coating or nail extension using either procedures relying on gel nail formulations or acrylic nail formulations.

One of the advantages of the present disclosure is a high viscosity composition which is easy to apply is provided. Compared to the low viscosity of nail gel formulations that may run into cuticles outside of the sculpting area, the compositions of the present disclosure have a high viscosity out of the container. The compositions of the present disclosure do not move easily until it is pushed into a desired shape by a manicurist tool, such as a brush or a pusher. The pushing or patting of the presently disclosed composition into a desired shape may be done neat, or it may be done with the aid of a patting liquid which aids in the patting of the composition into the desired shape. In at least some instances, the patting liquid may lower the viscosity of the composition or act as a plasticizer. One advantage is that either in the neat form or when used in contact with a patting liquid, the compositions of the present disclosure remain firm and do not run. The technician may optionally control the viscosity through the use of the suitable patting liquid applied until the composition of the present invention is cured by ultraviolet light. Thus, total control over the viscosity of the nail covering or nail extension may be attained.

Another advantage of the compositions of the present disclosure and the methods of using them over traditional acrylic nail formulations is that the presently disclosed compositions are one part formulations. There is no need to mix the compositions of the present disclosure with another part. By not spending time mixing the formulation or waiting for it to set to a workable viscosity, time serving the client is reduced, resulting in faster service. Further, the technician does not have to worry about getting the exact mixing ratios correct in order to get a workable composition.

Another advantage of the one-part formulation is the lack of heterogeneity in the formulations which may be associated with two-part formulations. The compositions of the present disclosure are homogeneous, allowing for uniform applicability of the composition.

A further advantage of the compositions of the present disclosure is a lack of odor of the one-part formulation. Traditional two-part acrylic systems typically exhibit very strong odors; for example, a common commercially available monomer composition that comprises a mixture of ethyl methacrylate, glycol HEMA-methacrylate, HEMA, benzophenone-1, and dimethyltolylamine, exhibits a severely strong odor. The composition of the present invention has little or no odor. This is especially desirable in small nail salons and spas, where multiple nail technicians may work side by side for many hours in a tight environment, or in environments with limited air circulation.

Yet a further advantage of the present disclosure is the lower exotherm profile compared to the builder gel formulations. UV light initiated builder gel formulas create heat when curing. Such heat can be unpleasant or even painful to the client. This is especially true if the product is applied in a thick layer, which results in an increase of heat flux from the gel into the nail bed. It has been observed that the compositions of the present disclosure exhibit a significantly lower exotherm of the polymerization reaction. Because of the lack of such a heat spike, the technician is able to apply the composition to the present invention in thicker layers when creating nail extensions. According to at least one aspect of the present disclosure, the rheology modifier of the presently disclosed compositions acts as a heat sink during the curing process. In some instances, the rheology modifier of the presently disclosed compositions is selected so as to modify the viscosity of the composition such that the exotherm profile is minimized while still providing suitable viscosity for shaping of the artificial nail or cosmetic nail coating.

A still further advantage of the present disclosure is the excellent adhesion, abrasion resistance, and mechanical strength of the cured nail coating. Another advantage of the present disclosure is that unlike gel nail coverings which tend to shrink while curing, the composition of the present disclosure displays no significant shrinkage.

A further advantage of the compositions of the present disclosure is the stability of the one-part formulation. Because the compositions are thermally stable and need UV light to cure it, the compositions of the present disclosure, at least under one embodiment, are shelf stable for several days, and at least under another embodiment are shelf stable for several months, and under at least one other embodiment are shelf stable for several years.

Yet another advantage of at least one embodiment of the present disclosure is the reduction of the yellowing of the cosmetic nail coating prepared from the compositions of the present disclosure. Unlike many other compositions that are thermally cured, or UV cured, the compositions of the present disclosure, when cured, do not appreciably yellow.

Yet still another advantage of the present disclosure compared to the traditional two-part system is the long open time to sculpt the nail. The presently disclosed compositions may be worked by the technician for as long as is necessary before the compositions are exposed to UV light.

The above advantages listed are illustrative and may not be exhaustive. Further, not every embodiment of the present disclosure necessarily displays all of the advantages listed.

The compositions of the present disclosure comprise at least one rheology modifier. According to at least one aspect of the present disclosure, the at least one rheology modifier may be selected in an amount such that the viscosity of the composition is greater than 400,000 centipoise (cps) at 25° C. as measured by a cone and plate rheometer using 40 mm 1° steel cone with a truncation gap of 31 microns at a 10 s⁻¹ shear rate on a 500 mg sample. In some instances, the at least one rheology modifier may be selected in an amount such that the viscosity of the composition is from about 600,000 cps to about 1,200,000 cps at 25° C. as measured by a cone and plate rheometer using 40 mm 1° steel cone with a truncation gap of 31 microns at a 10 s⁻¹ shear rate on a 500 mg sample. In at least some instances, the at least one rheology modifier may be selected in an amount such that the viscosity of the composition is from about 1,100,000 cps to about 3,250,000 cps at 25° C. as measured by a Brookfield Viscometer using spindle 96 at 0.3 rpm at the 1 minute mark. In some cases, the at least one rheology modifier may be selected in an amount such that the viscosity of the composition is from about 2,500,000 cps to about 3,150,000 cps at 25° C. as measured by a Brookfield Viscometer using spindle 96 at 0.3 rpm at the 1 minute mark.

According to at least one aspect of the present disclosure, the rheology modifier may be selected from the group consisting of methacrylate polymers, methacrylate copolymers, styrene polymers, styrene copolymers, polyethylene, polyesters, polysulfides, polycarbonates, silicones, carboxylic esters, and any combination thereof. The presently disclosed compositions may include from about 15 wt % to about 45 wt % of the at least one rheology modifier. The presently disclosed compositions may also include from about 20 wt % to about 35 wt % of the at least one rheology modifier.

In at least some instances, the presently disclosed compositions may include about 15 wt % to about 45 wt % of the at least one rheology modifier, wherein the rheology modifier comprises one or more polymers selected from the group consisting of poly(C₁₋₁₂alkyl(meth)acrylate) polymer, poly(C₁₋₁₂alkyl(meth)acrylate) copolymer, styrene polymer, styrene copolymer, and any mixture thereof. The compositions of the present disclosure may also comprise from about 15 wt % to about 45 wt % of a rheology modifier selected from the group consisting of poly(C₁₋₁₂alkylacrylate) polymer, poly(C₁₋₁₂alkylmethacrylate) polymer, poly(C₁₋₁₂alkylacrylate) copolymer, poly(C₁₋₁₂alkyl methacrylate) copolymer, styrene polymer, styrene copolymer, and mixtures thereof.

Under at least one embodiment of the present disclosure, the presently disclosed compositions comprise about 20 wt % to about 35 wt % of a rheology modifier comprising one or more poly(C₁₋₁₂alkyl(meth)acrylate) polymer.

In at least some instances, the rheology modifier may be a polymer that may be a powder that resembles the polymer powder that is typically used in the acrylic nail industry. The compositions of the present disclosure can comprise a polyalkyl(meth)acrylate, or a mixture thereof, wherein the alkyl groups comprise one to twelve carbon atoms.

In at least some instances, when mixed with the other ingredients of the photopolymerizable composition of the present disclosure, the polymer may not dissolve, but instead may form a homogenous suspension of the polymer in the composition.

The phrase “poly(C₁₋₁₂alkyl(meth)acrylate) polymer” means a polymer which is comprised mostly or exclusively of C₁₋₁₂alkyl(meth)acrylate monomer residues, or polymer units. Under one embodiment most or all of the C₁₋₁₂alkyl(meth)acrylate polymer units are the same. Under another embodiment, the C₁₋₁₂alkyl(meth)acrylate polymer units are different from each other. An example of “poly(Cₘ₂alkyl(meth)acrylate) polymer” includes a polymer that comprises methyl acrylate units, methacrylate units, and methyl methacrylate units.

The phrase “poly(C₁₋₁₂alkyl(meth)acrylate) copolymer” means a copolymer of C₁₋₁₂alkyl(meth)acrylate polymer units with other polymer units. The C₁₋₁₂alkyl(meth)acrylate polymer units may be the same or they may vary. The phrase “other polymer units” means polymer units that do not fall within the definition of poly(C₁₋₁₂alkyl(meth)acrylate); examples of such, include styrene, divinyl benzene, and a mixture of styrene and divinyl benzene.

Under one embodiment, the C₁₋₁₂alkyl(meth)acrylate polymer units in the poly(C₁₋₁₂alkyl(meth)acrylate) copolymer are the same units. Under another embodiment, the C₁₋₁₂alkyl(meth)acrylate polymer units in the poly(C₁₋₁₂alkyl(meth)acrylate) copolymer are different polymer units. An example of “poly(C₁₋₁₂alkyl(meth)acrylate) copolymer” includes a polymer that comprises methyl acrylate units, methyl methacrylate units, and styrene units.

The phrase “styrene polymer” means a polymer consisting essentially of —[CH₂—CHPh]— polymer units. The phrase “polymer unit” is synonymous with a “monomer residue,” or with a “repeating unit.”

The phrase “styrene copolymer” means a copolymer of styrene units with other polymer units that are not styrene. Examples of “styrene copolymer” includes styrene/(meth)acrylate copolymer, and styrene/(meth)acrylate/divinylbenzene copolymer. Copolymers comprising divinyl benzene are generally used in amounts as needed for crosslinking. Under at least one embodiment, the solid polymer comprises little or no crosslinking. Under another embodiment the solid polymer comprises a significant amount of crosslinking, which may mitigate the solubility of the polymer.

Examples of poly(C₁₋₁₂alkyl (meth)acrylate) include poly(methyl (meth)acrylate), poly(ethyl (meth)acrylate), poly(propyl (meth)acrylate), poly(n-propyl (meth)acrylate), poly(isopropyl (meth)acrylate), poly(butyl (meth)acrylate), poly(n-butyl (meth)acrylate), poly(isobutyl (meth)acrylate), poly(sec-butyl (meth)acrylate), poly(pentyl (meth)acrylate), poly(hexyl (meth)acrylate), poly(heptyl (meth)acrylate), poly(octyl (meth)acrylate), poly(nonyl (meth)acrylate), poly(decyl (meth)acrylate), poly(undecyl (meth)acrylate), and poly(dodecyl (meth)acrylate). In these examples, the alkyl groups hexyl, heptyl, octyl, nonyl, decyl, undecyl, and dodecyl may be straight chain groups or branched groups.

Under at least one embodiment of the present disclosure, the poly(C₁₋₁₂alkyl (meth)acrylate) polymer or copolymer thereof is a copolymer of poly(C₁₋₁₂alkyl (meth)acrylate). Examples of such copolymers include styrene/(meth)acrylic copolymer, styrene/(meth)acrylate, styrene/(meth)acrylate/divinylbenzene copolymers, and styrene/PEG-10 maleate/nonoxynol-10 maleate/acrylate copolymer.

Examples of poly(C₁₋₄alkyl (meth)acrylate) include poly(methyl (meth)acrylate), poly(ethyl (meth)acrylate), poly(propyl (meth)acrylate), poly(n-propyl (meth)acrylate), poly(isopropyl (meth)acrylate), poly(butyl (meth)acrylate), poly(n-butyl (meth)acrylate), poly(isobutyl (meth)acrylate), and poly(sec-butyl (meth)acrylate).

Under at least one embodiment of the present disclosure, the polymer consists of a single type of polymer. Under an alternative embodiment, the polymer consists of a mixture of poly(C₁₋₁₂alkyl acrylate) polymers; a mixture of poly(C₁₋₁₂alkyl methacrylate) polymers; a mixture of poly(C₁₋₁₂alkyl acrylate) polymer and poly(C₁₋₁₂alkyl methacrylate) polymer; a mixture of poly(C₁₋₁₂alkyl acrylate) polymers and poly(C₁₋₁₂alkyl methacrylate) polymer; a mixture of poly(C₁₋₁₂alkyl acrylate) polymers and poly(C₁₋₁₂alkyl methacrylate) polymer; a mixture of poly(C₁₋₁₂alkyl acrylate) polymers and poly(C₁₋₁₂alkyl methacrylate) polymers.

Examples of a suitable polymer comprises poly(methyl methacrylate), poly(ethyl methacrylate), and a mixture thereof. A suitable polymer may be poly(methyl methacrylate), or PMMA. Under one embodiment, PMMA is blended with other polymers to improve its flexibility. In some instances, the polymer may be a powder which may be prepared by a routine technique, such as suspension polymerization in which the reaction takes place between droplets of the corresponding monomer suspended in a solution of water and catalyst.

The molecular weight of the polymer suitable for use in the presently disclosed compositions and methods may be similar to the polymers used in the acrylic nail industry. The polymer powder prior to mixing with other ingredients may consist of fine microspheres. Under one embodiment, when mixed with the monomers and other components of the composition, these microspheres dissolve completely or partially.

Under another embodiment of the present disclosure, when mixed with the monomers and other components of the presently disclosed compositions, these microspheres are insoluble in the composition. Microspheres are said to be insoluble when less than 5% of the diameter of the microsphere is lost to the solution. Generally, insoluble microspheres gain weight and size as they swell upon exposure to the monomers and other ingredients.

According to at least one aspect of the present disclosure, the rheology modifier may comprise a polymer powder that consists of fine microspheres. Exemplary sizes of the microspheres (i.e., particle size of the powder) may be from about 1 μm to 100 μm. Under one embodiment of the present invention, the mean particle size of the rheology modifier polymer powder may be less than about 100 μm. Under another embodiment of the present disclosure, the mean particle size may be less than about 50 μm. Under still another embodiment of the present disclosure, the mean particle size may be less than about 10 μm. Under yet another embodiment of the present invention, the mean particle size may be less than about 5 μm.

According to at least one aspect of the present disclosure, the rheology modifier is a carboxylic ester. In some instances, the rheology modifier is a hydrogenated castor oil powder. In such instances, the hydrogenated castor oil powder may have an average particle size of from about 5 μm to about 10 μm.

According to at least one aspect of the present disclosure, the presently disclosed compositions may include from about 15 wt % to about 45 wt % of one or more binding agents. In at least some instances, the one or more binding agents may be selected from the group consisting of polyisobutene, ozokerite wax, ceresin, and a copolymer of acrylamide and sodium acryloyldimethyltaurate. In at least some instances, the binder may comprise a mixture of acrylamide, sodium acryloyldimethyl taurate copolymer, isohexadecane, and polysorbate 80. In other instances, the binder may comprise a mixture of polyacrylamide, C13-14 isoparaffin, and laureth-7. In some cases, the binder may be a copolymer of acrylamide and sodium acryloyldimethyltaurate. According to at least one aspect of the present disclosure, the one or more binding agents is not a wax. In some instances, the presently disclosed compositions exclude a wax. In some instances, the binder may exclude a wax.

In at least some instances, the presence of the binding agent in the presently disclosed compositions serves to hold all of the components of the formulation in a viscous state (semi-solid state) prior to curing. Upon curing, the binding agent or binding agents are substantially trapped within the crosslinks formed by the monomers rather than chemically reacting to form cross-links. In other words, the binding agents of the presently disclosed compositions are inert and therefore are trapped in the crosslinks of the composition upon curing. This is in contrast to artificial nail sculpting compositions or cosmetic nail coating compositions that include reactive oligomers which chemically react to form crosslinks.

The photopolymerizable composition of the present disclosure for forming a cosmetic coating for nails may also comprise about 15 wt % to about 45 wt % of one or more (meth)acrylate monomers. The (meth)acrylate monomer used in the present disclosure may be any acrylate monomer or methacrylate monomer that is used in nail art formulations in which the curing is performed by UV light. The (meth)acrylate monomer has a formula CH₂═C(R)—COOR′, wherein R is H, Me, or a mixture thereof, and R′ is an organic group. Examples of organic group R′ include hydrocarbons, that is, aliphatic (e.g., alkyl or alkenyl), alicyclic (e.g., cycloalkyl, cycloalkenyl) compounds, and aromatic-, aliphatic-, and alicyclic-substituted aromatic compounds, as well as cyclic compounds wherein the ring is completed through another portion of the molecule (e.g., two substituents together form an alicyclic compound); groups that include hetero atoms, that is, groups that contain other than carbon in a ring or chain otherwise composed of carbon atoms, such as oxygen, nitrogen, such as groups containing non-hydrocarbon groups, such as alkoxy, amino, amido, and similar groups.

Examples of (meth)acrylate include methyl acrylate, methyl methacrylate, ethyl acrylate, ethyl methacrylate, hydroxypropyl acrylate, hydroxypropyl methacrylate, butylacrylate, butyl methacrylate, hydroxyethyl acrylate, propyl methacrylate, isobutyl methacrylate, sec-butyl methacrylate, hydroxyethyl methacrylate, butoxyethyl acrylate, butoxyethyl methacrylate, diethylaminoethyl acrylate, diethylaminoethyl methacrylate, 2-ethylhexyl acrylate, 2-ethylhexyl methacrylate, ethoxyethyl acrylate, ethoxyethyl methacrylate, t-butyl aminoethyl acrylate, t-butyl aminoethyl methacrylate, methoxyethylene glycolacrylate, methoxyethylene glycol methacrylate, phosphoethyl acrylate, phosphoethyl methacrylate, methoxy propyl acrylate, methoxy propyl methacrylate, phenoxyethylene glycol acrylate, tetrahydrofurfuryl methacrylate, phenoxyethylene glycol methacrylate, caprolactone methacrylate, methacroyloxyethyl maleate, 2-hydroxyethyl methacrylate/succinate, phthalic acid monoethyl methacrylate, phenoxypolyethylene glycol acrylate, phenoxypolyethylene glycol methacrylate, 2-hydroxy-3-phenoxypropyl acrylate, 2-hydroxy-3-phenoxypropyl methacrylate, isobornyl acrylate, isobornyl methacrylate, 3-chloro-2-hydroxypropyl acrylate, 3-chloro-2-hydroxypropyl methacrylate, and mixtures thereof.

Under one embodiment of the present disclosure, the at least one or more (meth)acrylate monomers comprise a methacrylate selected from the group consisting of hydroxypropyl methacrylate, hydroxyethyl methacrylate, ethyl methacrylate, propyl methacrylate, butyl methacrylate, isobutyl methacrylate, sec-butyl methacrylate, tetrahydrofurfuryl methacrylate, caprolactone methacrylate, methacroyloxyethyl maleate, 2-hydroxyethyl methacrylate/succinate, phthalic acid monoethyl methacrylate, and any mixture thereof.

Under one embodiment of the present invention, the at least one of the one or more (meth)acrylate monomers comprises a hydroxyl-containing (meth)acrylate monomer.

The photopolymerizable compositions of the present disclosure for forming a cosmetic coating for nails may also optionally comprise one or more crosslinkers. A crosslinker is a compound that contains two or more (meth)acrylate groups. Crosslinkers are necessary to provide chemical bonding between several monomers, oligomers, polymers or combinations thereof to yield a cross-linked polymeric structure needed for the formation of cosmetic nail coating. Under one embodiment the monomer liquid comprises a single crosslinker. Under another embodiment the monomer liquid comprises two or more different compounds that are crosslinkers.

Examples of crosslinkers include diacrylates, triacrylates, tetraacrylates, pentaacrylates and higher acrylates. Such examples include trimethylolpropane triacrylate, trimethylolethane triacrylate, trimethylolpropane trimethacrylate, trimethylolethane trimethacrylate, tetramethylene glycol dimethacrylate, triethylene glycol dimethacrylate, tetraethylene glycol diacrylate, pentaerythritol diacrylate, penta-erythritol triacrylate, pentaerythritol tetraacrylate, dipentaerythritol diacrylate, dipenta-erythritol triacrylate, dipentaerythritol tetraacrylate, dipentaerythritol pentaacrylate, dipenta-erythritol hexaacrylate, tripentaerythritol octaacrylate, pentaerythritol dimethacrylate, penta-erythritol trimethacrylate, dipentaerythritol dimethacrylate, dipentaerythritol tetramethacrylate, tripentaerythritol octamethacrylate, ethylene glycol diacrylate, 1,3-butanediol diacrylate, 1,3-butanediol dimethacrylate, sorbitol triacrylate, sorbitol tetraacrylate, pentaerythritol-modified triacrylate, sorbitol tetramethacrylate, sorbitol pentaacrylate, sorbitol hexaacrylate, oligoester acrylates and methacrylates, glycerol di- and triacrylate, 1,4-cyclohexane diacrylate, bisacrylates and bismethacrylates of polyethylene glycol, and mixtures thereof.

Under one embodiment of the present disclosure, at least one of the one or more crosslinker may be selected from the group consisting of trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate and ethoxylated isocyanuric acid tri(meth)acrylates.

Under one embodiment of the present disclosure, the crosslinkers may comprise methacrylate groups. Example of such crosslinkers include dimethacrylates, trimethacrylates, tetramethacrylate, and higher methacrylates. Examples of such methacrylic crosslinkers include trimethylolpropane trimethacrylate, trimethylolethane trimethacrylate, tetramethylene glycol dimethacrylate, triethylene glycol dimethacrylate, pentaerythritol dimethacrylate, penta-erythritol trimethacrylate, dipentaerythritol dimethacrylate, dipentaerythritol tetramethacrylate, tripentaerythritol octamethacrylate, 1,3-butanediol dimethacrylate, sorbitol tetramethacrylate, oligoester methacrylates, bismethacrylates of polyethylene glycol having a molecular weight of from 200 to 1500, and mixtures thereof. For example, trimethylolpropane trimethacrylate is a composition consisting of, or comprising largely of, the compound of formula (CH₂═CMe-C(O)—O—CH₂)₃C—C₂H₅. It is a low volatility trifunctional monomer offering fast cure response in free radical polymerization.

Another suitable crosslinker is an alkoxylated crosslinker, with the formula (CH₂═CMe-C(O)—O-(AO)ₓ—CH₂—)₃C—R; wherein R is a C₁ to C₆ group with an ‘ene’ moeity; AO is a small alkoxy group, such as an ethylene oxide, —CH₂—CH₂—O—, propylene oxide, —CH(CH₃)—CH₂—O—, —CH₂—CH₂—CH₂—O—, butylene oxide, and —CH(Et)-CH₂—O—; and wherein for each (CH₂═CMe-C(O)—O-(AO)ₓ—CH₂—) group x is independently 0, 1, 2, or 3. Using R=ethyl, and AO=ethylene oxide as an example, an exemplary alkoxylated crosslinker has a structure of formula

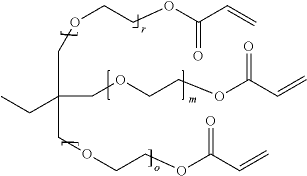

wherein m, n, and o are each independently 0, 1, 2, or 3.

Under one embodiment the cross linker in this composition is a tri((meth)acrylate), and the composition further comprises a photoinitiator.

In addition to the above-described (meth)acrylate-based polymerizable materials, other polymerizable monomers, oligomers or polymers of monomers which contain at least one free radical polymerizable group in the molecule may be used without any limitations in the curable gel. Typical examples include esters of acrylic and methacrylic acid, herein termed (meth)acrylic ester. Specific but not limiting examples of mono (meth)acryloyl esters include methyl (meth)acrylate, ethyl (meth)acrylate, hydroxypropyl (meth)acrylate, butyl (meth)acrylates, hydroxy ethyl (meth)acrylates, butoxyethyl (meth)acrylate, diethylaminoethyl (meth)acrylate, 2-ethylhexyl (meth)acrylate, ethoxyethyl (meth)acrylate, t-butyl aminoethyl (meth)acrylate, methoxyethylene glycol (meth)acrylate, phosphoethyl (meth)acrylate, methoxy propyl (meth)acrylate, methoxy polyethylene glycol(meth)acrylate, phenoxyethylene glycol (meth)acrylate, phenoxypolyethylene glycol (meth)acrylate, 2-hydroxy-3-phenoxypropyl (meth)acrylate, 2-(meth)acryloxyethylsuccinic acid, 2-(meth)acryloylethylphthalic acid, 2-(meth)acryloyloxypropylphthalic acid, stearyl (meth)acrylate, isobornyl (meth)acrylate, 3-chloro-2-hydroxypropyl (meth)acrylates, tetrahydrofurfuryl (meth)acrylate, methacryloyloxyethyl trimelilitc anhydride, (meth)acrylamides and allyl monomers. Specific but not limiting examples of difunctional (meth)acryloyl esters include 1,4 butane diol di(meth)acrylate, 1,6 hexananediol di(meth)acrylate, alkoxylated hexane diol di(meth)acrylate, 1,9 nonanediol di(meth)acrylate, 1,10 decanediol di(meth)acrylate, neopentyl glycol di(meth)acrylate, alkoxylated neopentyl glycol di(meth)acrylate, 2-methyl-1,8-octane diol di(meth)acrylate, cyclohexane dimethanol di(meth)acrylate, glycerin di(meth)acrylate, ethylene glycol di(meth)acrylate, triethyleneglycol di(meth)acrylate, polyethylene glycol di(meth)acrylate, propylene glycol di(meth)acrylate, polypropylene glycol di(meth)acrylate, ethoxylated propylene glycol di(meth)acrylate, ethoxylated polypropylene glycol di(meth)acrylate, polyethoxypropoxy di(meth)acrylate, ethoxylated bisphenol A di(meth)acrylate, propoxylated bisphenol A di(meth)acrylate, propoxylated ethoxylated bisphenol A di(meth)acrylate, bisphenol A glycidyl methacrylate, tricyclodecanedimethanol di(meth)acrylate, glycerin di(meth)acrylate, ethoxylated glycerin di(meth)acrylate, bis acrylamides, bis allyl ethers and allyl (meth)acrylates. Examples of tri and or higher (meth)acryloyl esters include trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate and ethoxylated isocyanuric acid tri(meth)acrylates. Monomers containing acid groups may also be used including (meth)acrylic acid, bis(glyceryl dimethacrylate) pyromellitate, pyromellitic dimethacrylate, methacryloyloxyethyl phthalate, methacryloyloxyethyl maleate, 2 hydroxyethyl methacrylate/succinate, 1,3 glyceryl dimethacrylate maleate adduct, and 1,3 glyceryl dimethacrylate succinate adduct. Partially aminated monomers and oligomers may also be used. These are prepared by reaction of amines, preferably secondary amines, with some of the (meth)acryloyl groups of the multifunctional monomers or oligomers.

The photopolymerizable composition of the present disclosure for forming a cosmetic coating for nails also comprises one or more photoinitiators. As described below, the photoinitiator may be selected so that it is activated by photons of the wavelength associated with UV light of the UV lamp. Preferably, the photoinitiator should be active at the wavelength of UV light of UV lamps commonly found in nail salons.

Such photoinitiators may be selected from benzyl ketones, monomeric hydroxyl ketones, polymeric hydroxyl ketones, α-amino ketones, acyl phosphine oxides, metallocenes, benzophenone, and benzophenone derivatives. Specific examples of photoinitiators include 1-hydroxy-cyclohexylphenylketone; benzophenone; 2-benzyl-2-(dimethylamino)-1-(4-(4-morphorliny Ophenyl)-1-butanone; 2,2-dimethoxy-2-phenyl acetophenone; 2-methy 1-1-(4-methylthio)phenyl-2-(4-morpholinyl)-1-propanone; 2,4,6-trimethylbenzoyldiphenyl-phosphine oxide; bis(2,4,6-trimethyl benzoyl)phenyl phosphine oxide; diphenyl-(2,4,6-trimethylbenzoyl) phosphine oxide; bis(2,6-dimethoxybenzoyl-2,4,4-trimethyl pentyl)phosphine oxide; 2-hydroxy-2-methyl-1-phenyl-propan-1-one; phenyl bis(2,4,6-trimethylbenzoyl) phosphine oxide; benzyl-dimethylketal; isopropylthioxanthone; bis(η⁵-2,4-cyclopentadien-1-yl)bis[2,6-difluoro-3-(1H-pyrrol-1-yl)phenyl]titanium), and mixtures of any of the foregoing.

Under one embodiment of the present disclosure the photopolymerizable composition comprises a single chemical compound that exhibits photoinitiating properties. Under an alternative embodiment of the present disclosure, the photoinitiator is a mixture of photoinitiators.

The photoinitiator may be present in the photopolymerizable composition at amounts sufficient to be effective in aiding curing of the photopolymerizable composition. Such amounts may be determined empirically. The photopolymerizable composition may comprise up to about 10 wt % of one or more photoinitiators. Under one embodiment, the photopolymerizable composition may comprise about 0.5 to about 5.0 wt % of one or more photoinitiators.

The photopolymerizable composition of the present invention under one embodiment may further comprise a small amount of a colorant or special effects pigment or a combination thereof.

One purpose of using pigment in the photopolymerizable composition is to provide a tint or a color to the formed cosmetic nail coating. The use of such color in the photopolymerizable composition may allow the technician to omit certain selected post treatment steps after the formation of the cosmetic nail coating.

Another purpose of using a pigment is to give a clear or colorless or whitish appearance of the cosmetic nail coating. The pigment may be used to address any yellowing of the cosmetic nail coating. Yet another purpose of using a pigment is to provide a whitish appearance to the photopolymerizable composition, so that it appears as an attractive, clean product to the nail technician.

Examples of pigments that may be incorporated into the photopolymerizable composition of the present disclosure include: annatto, caramel, carmine, B-carotene, potassium sodium copper chlorophyllin (chlorophyllin copper-complex), dihydroxyacetone, bismuth oxychloride, guaiazulene, iron oxides, ferric ammonium ferrocyanide, ferric ferrocyanide, chromium hydroxide green, chromium oxide greens, guanine, pyrophyllite, mica, silver, titanium dioxide, aluminum powder, bronze powder, copper powder, ultramarines, manganese violet, zinc oxide, luminescent zinc sulfide, FD&C Blue No. 1, D&C Blue No. 4, Iron Blue, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, and any mixture of the foregoing. As will be recognized by the practitioner of the art, some of the pigments in the above list are better suited for use in the photopolymerizable composition than others, because they offer better composition stability of the photopolymerizable composition, and they do not interfere with the UV curing process.

Under at least one embodiment of the present disclosure, the photopolymerizable composition comprises a pigment is selected from the group consisting of ultramarine, manganese violet, zinc oxide, FD&C Blue No. 1, D&C Blue No. 4, Iron Blue, D&C Violet No. 2, and any mixture thereof.

Special effects pigment may be any pigment that gives either the photopolymerizable composition or the formed cured composition a special effect, such as an increased pearlescent, iridescent, shimmering, transparency or complex effects. Examples of special effect pigments include titanated micas, mica based interference colors, mica coated with titanium dioxide and iron oxide, mica based gold pearls, mica based metallic pearls, mica based pearl pigments, bismuth oxychloride, synthetic mica based interference pearls, synthetic mica based white pigment, silicate based pearls, titanium oxide and tin oxide on silicate platelets, flaked aluminum powder, silver coated silicate flakes, and any combination of the foregoing.

The photopolymerizable composition of the present disclosure may further comprise additional ingredients, including colorants, dyes, whiteners, perfumes, thixotropes, stabilizers, anti-oxidants, and the like. Any of above polymers, oligomers, monomers, crosslinkers, pigments, and other ingredients as provided by its manufacturer may contain small amounts of additives and impurities. The purity level of the ingredient may be above 90%. Alternatively, the purity level may be above 95%. Under some embodiments the purity level may be above 97%. Additives for monomers may include inhibitors such as hydroquinone, HQ, monomethyl ether quinone, MEHQ, isoascorbic acid, IA, butylated hydroxytoluene, BHT, and BHT adducts. Impurities may include isomers of the monomer, oligomers, unreacted starting materials, water, and solvent used in the formation of the monomers.

According to at least one aspect of the present disclosure, the presently disclosed compositions do not include reactive oligomers that may be found in other sculpting compositions. In this aspect, the presently disclosed compositions include only inert polymers rather than the reactive oligomers and reactive polymers relied upon in other sculpting compositions.

The photopolymerizable compositions of the present disclosure for forming a cosmetic coating for nails is a viscous liquid, such as a gel. It has a similar consistency as a mixture of polymer powder and monomer liquid in a traditional two-part system that is middle of the open time.

Under one embodiment of the present disclosure, the viscosity of the photopolymerizable composition is greater than about 400,000 centipoise as measured by a cone and plate rheometer. The viscosity is determined by a cone and plate rheometer using 40 mm 1° steel cone with the truncation gap of 31 microns operating at 2 to 10 s⁻¹ shear rate on a 500 mg sample. The temperature of the Peltier plate is set 25° C., and the sample is conditioned to 25° C. The cone and plate rheometer may be any suitable rheometer for measuring high viscosity fluids, such as TA Instruments AR1500EX Rheometer. The viscosity is determined from measurements at 5 points from 2 s⁻¹ to 10 s⁻¹ shear rates.

Under one embodiment of the present disclosure, the viscosity of the compositions may be from about 600,000 centipoise to about 1,200,000 centipoise. In at least some instances, the viscosity of the composition may be from about 1,100,000 cps to about 3,250,000 cps at 25° C. as measured by a Brookfield Viscometer using spindle 96 at 0.3 rpm at the 1 minute mark. In other instances, the viscosity of the composition may be from about 2,500,000 cps to about 3,150,000 cps at 25° C. as measured by a Brookfield Viscometer using spindle 96 at 0.3 rpm at the 1 minute mark.

The present disclosure is also directed to photopolymerizable compositions wherein the compositions exhibit a lower exotherm. The exotherm is the apparent rise in temperature due to the heat generated during the curing reaction caused by UV light. The exotherm may be measured on a 0.5 gram sample formed on a glass substrate into a shape approximating a sculpted nail. The exotherm may be measured by a thermocouple on the glass substrate to measure the approximate rise in temperatures that a client would feel during the curing process under the UV light.

The exotherm may be decreased by increasing the wt % of the rheology modifier of the presently disclosed compositions. Further, the exotherm may be decreased by decreasing the wt % of the photoinitiators.

Under one embodiment of the present disclosure, the exotherm (i.e., the peak temperature achieved during the reaction less the starting temperature) is less than about 30° C. (54° F.). Under another embodiment of the present disclosure, the exotherm is less than about 20° C. (36° F.). Under still another embodiment of the present disclosure, the exotherm is less than about 15° C. (27° C.).

The photopolymerizable compositions of the present disclosure under at least one embodiment is stable in a dark container at 49° C. for four months, or at 65° C. for 2 weeks. The stability may be tested in an enclosed container that does not let any UV light in. The term “stable” refers to the lack of change in the physical, chemical or aesthetic properties of the photopolymerizable composition that would make the photopolymerizable composition unsuitable for sale to the consumer or would unsuitable for use by the technician. Exemplary changes of physical, chemical or aesthetic properties of the photopolymerizable composition include polymerization, noticeable yellowing, or a noticeable increase in viscosity.

The photopolymerizable compositions of the present disclosure may be prepared by any means used to add and blend high viscosity compositions. The components may be added together in any order that is convenient from an engineering viewpoint. Addition of the components, blending of the components and filling appropriate containers may be done at elevated temperatures. Such temperatures should be less than temperatures which would initiate curing of the components.

The container in which the photopolymerizable composition of the present disclosure is sold may be any container capable of handling high viscosity compositions and of providing protection from UV light. One example of such a container is a wide-mouth jar. Another example of a suitable container is a tube container. Another example of a suitable container is a syringe container.

In another aspect of the present disclosure, a container is provided comprising the previously described photopolymerizable compositions, wherein the container is a tube container or a syringe container, and wherein the container delivers a uniform bead of the composition via an orifice of the container.

A tube container of the present disclosure may comprise the previously described photopolymerizable compositions inside of a tube. The tube may be a cylindrical, hollow piece with a round or oval profile, similar to tubes used to contain toothpaste, ointment, adhesives, caulk, and other viscous liquids. The tube has an orifice which is designed to deliver a bead of the photopolymerizable composition. Such an orifice may be a part of a nozzle, which may be optionally capped or closed.

A syringe container of the present disclosure may comprise the previously described photopolymerizable compositions in a syringe. The syringe of the present disclosure may comprise a plunger that fits tightly into a barrel. The plunger can be pushed along inside the barrel, allowing the syringe to expel the photopolymerizable composition through an orifice at the open end of the tube. The orifice is designed to deliver a bead of the photopolymerizable composition. Such an orifice may be a part of a nozzle, which may be optionally capped or closed.

The orifice may be designed so that the technician is able to deliver a uniform bead to the nail of the client. The phrase “uniform bead” means that the weight of the bead formed does not vary by more than 10 wt % from another bead when forced through the orifice using the same conditions (the same pressure and same length of time) as subjectively applied by the technician.

In another aspect of the present disclosure, a method of using the above-described photopolymerizable compositions to form a cosmetic coating for a nail is provided. In particular, the present disclosure provides a method of forming a cosmetic nail or an artificial nail, the method comprising: (a) contacting the presently disclosed composition with a patting liquid selected from the group consisting of an acrylic nail monomer, solvent, oil, slip agent, photoinitiator, and any mixture thereof, to form a blend; (b) placing the blend onto a nail; and (c) exposing the blend to UV light. The present disclosure also provides a method of forming a cosmetic nail or an artificial nail, the method comprising (a) placing the presently disclosed composition onto a nail; (b) contacting the composition with a patting liquid selected from the group consisting of an acrylic nail monomer, solvent, oil, photoinitiator, and any mixture thereof; and (c) exposing the mixture to UV light. The phrase “acrylic nail monomer” or the phrase “monomer liquid” means a liquid that is used by technicians to prepare acrylic nails. The term “liquid” when referring to the monomer liquid means a liquid composition that is sufficiently homogeneous to be used for preparation of acrylic nails. The liquid may be a uniform clear solution, a liquid that displays a Tyndall effect, a colloid, or a suspension in which any fine particles of the suspension do not precipitate during storage time. The liquid may be colorless, or it may be colored.

The present disclosure also provides a method for forming a cosmetic nail coating or an artificial nail, the method comprising: applying a composition onto a nail in one application step, the composition comprising: (a) from about 15 wt % to about 45 wt % of at least one rheology modifier; (b) from about 25 wt % to about 75 wt % of one or more (meth)acrylate monomers; and (c) one or more photoinitiators; and exposing the composition to UV light; wherein the composition is in the form of a liquid or gel and is not mixed with any additional components prior to the applying of the composition onto the nail.

In the course of performing the above-described methods, the placement of the bead of the photopolymerizable composition may be performed by a technician directly by squeezing it from a tube container or pushing it from a syringe with a plunger. Alternatively, the bead of the photopolymerizable composition may be done with the help of an acrylic or gel brush, such as a No. 8, 10, or 12 brush. After the placement of the bead of the photopolymerizable composition onto the nail, the technician works the composition to move it into a desired location and form it into a desired shape.

After the composition is shaped, the composition, along with finger and the hand of the client, is exposed to UV light to cure the composition. A suitable UV light may be natural sunlight. Another suitable UV light may be a UV lamp, such as a 36 watt lamp commonly used in many nail salons. Such a UV lamp may operate at any wavelength required to cure the photopolymerizable composition, such as between 320 nm and 420 nm. The exposure time should be as long enough to allow for curing of the photopolymerizable composition. This may be 5 seconds to 6 minutes.

The term “UV lamp” is meant to be interpreted broadly. It refers to any source of electromagnetic radiation that exhibits light in the 320 nm to 420 nm range at sufficient enough strength to cure the composition of the present disclosure. The term “UV lamp” includes traditional UV lamps that contain fluorescent lamps, such as compact fluorescent light bulbs, that give off UV light in the above-described ranges. The term “UV lamp” also refers to newer sources of light or UV radiation, such as light-emitting diode lamps (commonly referred to as “LED lamps”) that emit electromagnetic radiation which includes UV light in the 320 nm to 420 nm range at sufficient enough strength to cure the composition of the present disclosure. The term “UV lamp” also refers to any other type of source of light that comprises UV light in the 320 nm to 420 nm range at sufficient enough strength to cure the composition of the present disclosure

The terms “cure”, “curing”, and like, are used herein are similar to those in the nail art, and are broadly encompassing terms. These terms refer to any portion of, or the entire process of polymerization which is experienced under the UV light.

### Examples

Exemplary photopolymerizable compositions for forming a cosmetic coating for nails have the following ingredients:

Formulation 1

| Chemical name | wt % |
|---|---|
| hydrogenated castor oil powder | 15-30 |
| Mixture of hydroxyalkyl | 50-60 |
| (meth)acrylate and cycloalkyl | |
| (meth)acrylate monomers | |
| copolymer of acrylamide and | 25-35 |
| sodium acryloyldimethyltaurate | |
| Photoinitiator | 2 |
| Antioxidant | <1 |
| Colorant | <0.05 |

Formulation 2

| Chemical name | wt % |
|---|---|
| (Meth)acrylic polymer rheology modifier | 20-25 |
| acrylamide or polyacrylamide-based | 25-35 |
| binding agent | |
| Mixture of hydroxyalkyl | 50-65 |
| (meth)acrylate and cycloalkyl | |
| (meth)acrylate monomers | |
| Photoinitiator | 2 |
| Antioxidant | <1 |
| Colorant | <0.05 |

Formulation 3

| Chemical name | wt % |
|---|---|
| carboxylic ester rheology modifier | 15-35 |
| acrylamide or polyacrylamide-based | 15-35 |
| binding agent | |
| Mixture of hydroxyalkyl | 45-65 |
| (meth)acrylate and cycloalkyl | |
| (meth)acrylate monomers | |
| Photoinitiator | 2 |
| Antioxidant | <1 |
| Colorant | <0.05 |

For each of the formulations, the above ingredients were added together and mixed until the composition was homogeneous. The order of addition of the ingredients did not show any appreciable differences in the performance of the composition.

A sample bead of the above formulation was placed on a microscope slide. The bead had the viscosity and workability of a typical traditional two part system about one minute after the monomer liquid and the polymer have been mixed. The open time appeared to be much longer than typically necessary by even the most inexperienced nail technician. The bead was spread and exposed to a UV lamp, to yield a hard, durable substance.

### Viscosity Measurements

The viscosities of samples of the above formulation (prior to curing) were measured using a TA Instruments AR1500EX Rheometer using a cone and plate setup, with a 40 mm 1° steel cone. The temperature of the Peltier plate was set 25° C. and the instrument was calibrated prior the use. The sample (0.5±0.05 g) was loaded in the center of the plate, the head was lowered to the truncation gap of 31 microns, and the sample was conditioned to 25° C. The measurements were taken under steady state flow conditions, where the temperature and truncation gap were held constant throughout the run. The viscosity of the samples was measured at 5 points during the analysis, with each data point collected at a different, increasing shear rate. The initial shear rate is 2 s⁻¹ and each subsequent measurement was taken as the shear rate was increased by increments of 2 s⁻¹ until the final shear rate of 10 s⁻¹ was reached. The viscosity obtained at the 10 s⁻¹ shear rate was recorded as the final viscosity of the sample. The viscosity of a typical sample of Formulations 1-3 was from about 600,000 to about 1,200,000 cps at 25° C. as measured by a Brookfield Viscometer using spindle 96 at 0.3 rpm at the 1 minute mark.

### Exotherm Measurements

A 0.5 g sample of the above formulations was compared to a 0.5 g sample of a mixture of the same gel and a polymer powder, and the amount of photoinitiators was adjusted so that both samples had the same quantity of photoinitiators. The samples were applied to a thermocouple on a glass slide. After photoinitiation, there was a 19° C. rise in the sample without the polymer powder but only a 12° C. rise in the sample with the polymer powder.

### Stability Testing

An accelerated thermal stability test was completed on samples of three formulations of a one-part acrylic nail formula of the present invention. The samples were monitored at various time intervals and compared to one another to observe if any changes in physical state or performance of the samples could be noted; namely yellowing and polymerization. Under this test, four months at 49° C. and two weeks at 65° C. are the approximate equivalent to having a sample age two years at room temperature; any sample that remained relatively unchanged after 2 weeks at 65° C. was said to pass stability testing.

The samples were contained in ½ oz (15 mL) polypropylene containers fitted with a foil seal lid with a 15% head space and were monitored at 0 days, 1 day, 3 days, 1 week, and 2 weeks for any observable differences at room temperature, 49° C., and 65° C. Formulations 1, 2 and 3 remained stable over the 2 week observation period at room temperature, 49° C., and 65° C.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

### Statements of the Disclosure Include

Statement 1: A photopolymerizable composition comprising: (a) from about 15 wt % to about 45 wt % of at least one rheology modifier; (b) from about 25 wt % to about 75 wt % of one or more (meth)acrylate monomers; and (c) one or more photoinitiators.

Statement 2: A photopolymerizable composition according to Statement 1, further comprising (d) one or more binding agents.

Statement 3: A photopolymerizable composition according to Statement 2, wherein the composition comprises from about 15 wt % to about 45 wt % of the one or more binding agents.

Statement 4: A photopolymerizable composition according to Statement 2 or Statement 3, wherein the one or more binding agents comprises one or more selected from the group consisting essentially of polyisobutene, ozokerite wax, ceresin, and a copolymer of acrylamide and sodium acryloyldimethyltaurate.

Statement 5: A photopolymerizable composition according to Statement 2 or Statement 3, wherein the one or more binding agents comprises acrylamide, sodium acryloyldimethyl taurate copolymer, isohexadecane, polysorbate 80, and any combination thereof.

Statement 6: A photopolymerizable composition according to Statement 2 or Statement 3, wherein the one or more binding agents comprises polyacrylamide, C13-14 isoparaffin, laureth-7, and any combination thereof.

Statement 7: A photopolymerizable composition according to Statement 2 or Statement 3, wherein the one or more binding agents is not a wax.

Statement 8: A photopolymerizable composition according to Statement 2 or Statement 3, wherein the one or more binding agents excludes a wax.

Statement 9: A photopolymerizable composition according to any one of the preceding Statements 1-8, wherein the composition excludes a wax.

Statement 10: A photopolymerizable composition according to any one of the preceding Statements 2-9, wherein the composition comprises from about 15 wt % to about 35 wt % of the one or more binding agents.

Statement 11: A photopolymerizable composition according to any one of the preceding Statements 1-10, wherein the at least one rheology modifier is selected in an amount such that the viscosity of the composition is greater than 400,000 centipoise (cps) at 25° C. as measured by a cone and plate rheometer using 40 mm 1° steel cone with a truncation gap of 31 microns at a 10 s⁻¹ shear rate on a 500 mg sample.

Statement 12: A photopolymerizable composition according to any one of the preceding Statements 1-10, wherein the at least one rheology modifier is selected in an amount such that the viscosity of the composition is from about 600,000 cps to about 1,200,000 cps at 25° C. as measured by a cone and plate rheometer using 40 mm 1° steel cone with a truncation gap of 31 microns at a 10 s⁻¹ shear rate on a 500 mg sample.

Statement 13: A photopolymerizable composition according to any one of the preceding Statements 1-10, wherein the at least one rheology modifier is selected in an amount such that the viscosity of the composition is from about 1,100,000 cps to about 3,250,000 cps at 25° C. as measured by a Brookfield Viscometer using spindle 96 at 0.3 rpm at the 1 minute mark.

Statement 14: A photopolymerizable composition according to any one of the preceding Statements 1-10, wherein the at least one rheology modifier is selected in an amount such that the viscosity of the composition is from about 2,500,000 cps to about 3,150,000 cps at 25° C. as measured by a Brookfield Viscometer using spindle 96 at 0.3 rpm at the 1 minute mark.

Statement 15: A photopolymerizable composition according to any one of the preceding Statements 1-14, wherein the at least one rheology modifier is selected from the group consisting of methacrylate polymers, methacrylate copolymers, styrene polymers, styrene copolymers, polyethylene, polyesters, polysulfides, polycarbonates, silicones, carboxylic esters, and any combination thereof.

Statement 16: A photopolymerizable composition according to any one of the preceding Statements 1-14, wherein the at least one rheology modifier is a carboxylic ester.

Statement 17: A photopolymerizable composition according to any one of the preceding Statements 1-14, wherein the at least one rheology modifier is a hydrogenated castor oil powder.

Statement 18: A photopolymerizable composition according to Statement 17, wherein the hydrogenated castor oil powder comprises an average particle size of from about 5 μm to about 10 μm.

Statement 19: A photopolymerizable composition according to any one of the preceding Statements 1-18, wherein the composition comprises from about 20 wt % to about 35 wt % of the at least one rheology modifier.

Statement 20: A photopolymerizable composition according to any one of the preceding Statements 1-19, wherein the rheology modifier comprises a polymer that is insoluble in the composition.

Statement 21: A photopolymerizable composition according to any one of the preceding Statements 1-20, wherein the rheology modifier comprises a polymer that forms a suspension of the polymer in the composition.

Statement 22: A photopolymerizable composition according to any one of the preceding Statements 1-21, wherein the at least one rheology modifier comprises a mean particle size of less than about 100 micrometers.

Statement 23: A photopolymerizable composition according to any one of the preceding Statements 1-21, wherein the at least one rheology modifier comprises a mean particle size of less than about 50 micrometers.

Statement 24: A photopolymerizable composition according to any one of the preceding Statements 1-21, wherein the at least one rheology modifier comprises a mean particle size of less than about 10 micrometers.

Statement 25: A photopolymerizable composition according to any one of the preceding Statements 1-21, wherein the at least one rheology modifier comprises a mean particle size of less than about 5 micrometers.

Statement 26: A photopolymerizable composition according to any one of the preceding Statements 1-25, wherein the rheology modifier is at least in part present in the composition as insoluble microspheres.

Statement 27: A photopolymerizable composition according to any one of the preceding Statements 2-26, wherein upon curing the composition by exposure to ultraviolet light, the one or more binding agents are trapped in crosslinks.

Statement 28: A photopolymerizable composition according to any one of the preceding Statements 2-27, wherein the one or more binding agents are inert and do not chemically react to form crosslinks prior to or after curing of the composition by exposure to ultraviolet light.

Statement 29: A photopolymerizable composition according to any one of the preceding Statements 1-28, wherein the composition excludes reactive oligomers and reactive polymers.

Statement 30: A photopolymerizable composition according to any one of the preceding Statements 1-29, wherein the composition only includes inert polymers.

Statement 31: A photopolymerizable composition according to any one of the preceding Statements 1-30, wherein the one or more (meth)acrylate monomers comprises a hydroxyl-containing (meth)acrylate monomer.

Statement 32: A photopolymerizable composition according to any one of the preceding Statements 1-31, wherein the one or more (meth)acrylate monomers comprises a methacrylate selected from the group consisting of hydroxypropyl methacrylate, hydroxyethyl methacrylate, ethyl methacrylate, propyl methacrylate, butyl methacrylate, isobutyl methacrylate, sec-butyl methacrylate, tetrahydrofurfuryl methacrylate, caprolactone methacrylate, methacroyloxyethyl maleate, 2-hydroxyethyl methacrylate/succinate, phthalic acid monoethyl methacrylate, and any mixture thereof.

Statement 33: A photopolymerizable composition according to any one of the preceding Statements 1-32, further comprising (e) one or more crosslinkers.

Statement 34: A photopolymerizable composition according to Statement 33, wherein at least one of the one or more crosslinkers is selected from the group consisting of tri(meth)acrylate, tetra(meth)acrylate, penta(meth)acrylate, and any mixture thereof.

Statement 35: A photopolymerizable composition according to Statement 33, wherein at least one of the one or more crosslinkers is an alkoxylated crosslinker having the formula (CH₂═CMe-C(O)—O-(AO)ₓ—CH₂—)₃C—R wherein R is a C₁ to C₆ group with an ‘ene’ moiety, AO is an alkoxy group selected from the group consisting of ethylene oxide, —CH₂—CH₂—O—, propylene oxide, —CH(CH₃)—CH₂—O—, —CH₂—CH₂—CH₂—O—, butylene oxide, and —CH(Et)-CH₂—O—; and wherein for each CH₂═CMe-C(O)—O-(AO)ₓ—CH₂— group x is independently 0, 1, 2, or 3.

Statement 36: A photopolymerizable composition according to Statement 33, wherein at least one of the one or more crosslinkers is an ethoxylated crosslinker having the formula (CH₂═CMe-C(O)—O—(CH₂—CH₂—O)ₓ—CH₂)₃C—C₂H₅; wherein for each CH₂═CMe-C(O)—O—(CH₂—CH₂—O)ₓ—CH₂— group x is independently 0, 1, 2, or 3.

Statement 37: A photopolymerizable composition according to Statement 33, wherein at least one of the one or more crosslinkers is selected from the group consisting of trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate, and ethoxylated isocyanuric acid tri(meth)acrylates.

Statement 38: A photopolymerizable composition according to Statement 33, wherein the one or more crosslinkers comprises tri(meth)acrylate.

Statement 39: A photopolymerizable composition according to any one of the preceding Statements 1-38, wherein at least one of the one or more photoinitiators is selected from the group consisting of 1-hydroxy-cyclohexylphenylketone, benzophenone, 2-benzyl-2-(dimethylamino)-1-(4-(4-morpholinyl)phenyl)-1-butanone, 2,2-dimethoxy-2-phenyl acetophenone, 2-methyl-1-(4-methylthio)phenyl-2-(4-morpholinyl)-1-propanone, 2,4,6-trimethylbenzoyldiphenyl-phosphine oxide, bis(2,4,6-trimethyl benzoyl)phenyl phosphine oxide, diphenyl-(2,4,6-trimethylbenzoyl) phosphine oxide, bis(2,6-dimethoxybenzoyl-2,4,4-trimethyl pentyl)phosphine oxide, 2-hydroxy-2-methyl-1-phenyl-1-propanone, phenyl bis(2,4,6-trimethylbenzoyl) phosphine oxide, benzyl-dimethylketal, isopropylthioxanthone, bis(η⁵-2,4-cyclopentadien-1-yl)bis[2,6-difluoro-3-(1H-pyrrol-1-yl)phenyl]titanium), α,α-dimethoxy α-phenyl acetophenone, ethyl (2,4,6-trimethyl benzoyl) phenyl phosphinate, phenyl (2,4,6-trimethyl benzoyl) phenyl phosphinate, methyl benzoyl formate, and any mixture thereof.

Statement 40: A photopolymerizable composition according to any one of the preceding Statements 1-39, wherein the composition comprises from about 0.5 wt % to about 5.0 wt % of the one or more photoinitiators.

Statement 41: A photopolymerizable composition according to any one of the preceding Statements 1-40, further comprising a colorant, a special effects pigment, or a combination thereof.

Statement 42: A photopolymerizable composition according to any one of the preceding Statements 1-41, wherein the colorant is selected from the group consisting of ultramarine, manganese violet, zinc oxide, FD&C Blue No. 1, D&C Blue No. 4, Iron Blue, D&C Violet No. 2, and any mixture thereof.

Statement 43: A photopolymerizable composition according to any one of the preceding Statements 1-42, wherein the rheology modifier is selected in an amount such that a 0.5 gram sample of the composition exhibits a temperature increase of less than about 20° C. during the exposure to UV light.

Statement 44: A photopolymerizable composition according to any one of the preceding Statements 1-43, wherein the composition is stable in a dark container at 49° C. for four months or at 65° C. for 2 weeks.

Statement 45: A photopolymerizable composition according to any one of the preceding Statements 1-44, wherein the composition comprises from about 35 wt % to about 75 wt % of the one or more (meth)acrylate monomers.

Statement 46: A photopolymerizable composition according to any one of the preceding Statements 1-45, wherein the composition is a liquid or gel capable of forming an artificial nail or cosmetic nail coating in one application and without being mixed with any additional components.

Statement 47: A photopolymerizable composition according to any one of the preceding Statements 1-46, wherein the container is a tube container or a syringe container, and wherein the container delivers a uniform bead of the composition.

Statement 48: An artificial nail or cosmetic nail coating kit comprising: at least one first container containing an artificial nail sculpting composition according to any one of the preceding Statements 1-46; and at least one second container containing a patting solution for contacting with the artificial nail sculpting composition to aid the spreading and shaping of the artificial nail sculpting composition on a nail.

Statement 49: An artificial nail or cosmetic nail coating kit according to Statement 48, wherein the first container is a tube container or a syringe container, and wherein the first container delivers a uniform bead of the artificial nail sculpting composition to a nail.

Statement 50: An artificial nail or cosmetic nail coating kit according to Statement 48 or Statement 49, wherein the patting solution is selected from the group consisting of an acrylic nail monomer, solvent, oil, slip agent, photoinitiator, and any mixture thereof.

Statement 51: An artificial nail or cosmetic nail coating kit according to any one of the preceding Statements 48-50, further comprising at least one brush for contacting the patting solution of the second container with the builder composition of the first container after the artificial nail sculpting composition is applied to a nail.

Statement 52: A method for forming a cosmetic nail coating or an artificial nail, the method comprising: applying a composition onto a nail in one application step, the composition comprising: (a) from about 15 wt % to about 45 wt % of at least one rheology modifier; (b) from about 25 wt % to about 75 wt % of one or more (meth)acrylate monomers; and (c) one or more photoinitiators; exposing the composition to UV light; wherein the composition is in the form of a liquid or gel and is not mixed with any additional components prior to the applying of the composition onto the nail.

Statement 53: A method for forming a cosmetic nail coating or an artificial nail according to Statement 52, wherein the composition further comprises (d) one or more binding agents.

Statement 54: A method for forming a cosmetic nail coating or an artificial nail according to Statement 53, wherein the composition comprises from about 15 wt % to about 45 wt % of the one or more binding agents.

Statement 55: A method for forming a cosmetic nail coating or an artificial nail according to Statement 53 or Statement 54, wherein the one or more binding agents comprises one or more selected from the group consisting essentially of one or more selected from the group consisting essentially of polyisobutene, ozokerite wax, ceresin, and a copolymer of acrylamide and sodium acryloyldimethyltaurate.

Statement 56: A method for forming a cosmetic nail coating or an artificial nail according to Statement 53 or Statement 54, wherein the one or more binding agents comprises acrylamide, sodium acryloyldimethyl taurate copolymer, isohexadecane, polysorbate 80, and any combination thereof.

Statement 57: A method for forming a cosmetic nail coating or an artificial nail according to Statement 53 or Statement 54, wherein the one or more binding agents comprises polyacrylamide, C13-14 isoparaffin, laureth-7, and any combination thereof.

Statement 58: A method for forming a cosmetic nail coating or an artificial nail according to any one of the preceding Statements 53-57, wherein the one or more binding agents is not a wax.

Statement 59: A method for forming a cosmetic nail coating or an artificial nail according to any one of the preceding Statements 53-57, wherein the one or more binding agents excludes a wax.

Statement 60: A method for forming a cosmetic nail coating or an artificial nail according to any one of the preceding Statements 52-57, wherein the composition excludes a wax.

Statement 61: A method for forming a cosmetic nail coating or an artificial nail according to any one of the preceding Statements 53-60, wherein the composition comprises from about 15 wt % to about 35 wt % of the one or more binding agents.

Statement 62: A method for forming a cosmetic nail coating or an artificial nail according to any one of the preceding Statements 52-57, wherein the at least one rheology modifier is selected in an amount such that the viscosity of the composition is greater than 400,000 centipoise (cps) at 25° C. as measured by a cone and plate rheometer using 40 mm 1° steel cone with a truncation gap of 31 microns at a 10 s⁻¹ shear rate on a 500 mg sample.

Statement 63: A method for forming a cosmetic nail coating or an artificial nail according to any one of the preceding Statements 52-57, wherein the at least one rheology modifier is selected in an amount such that the viscosity of the composition is from about 600,000 cps to about 1,200,000 cps at 25° C. as measured by a cone and plate rheometer using 40 mm 1° steel cone with a truncation gap of 31 microns at a 10 s⁻¹ shear rate on a 500 mg sample.

Statement 64: A method for forming a cosmetic nail coating or an artificial nail according to any one of the preceding Statements 52-57, wherein the at least one rheology modifier is selected in an amount such that the viscosity of the composition is from about 1,100,000 cps to about 3,250,000 cps at 25° C. as measured by a Brookfield Viscometer using spindle 96 at 0.3 rpm at the 1 minute mark.

Statement 65: A method for forming a cosmetic nail coating or an artificial nail according to any one of the preceding Statements 52-57, wherein the at least one rheology modifier is selected in an amount such that the viscosity of the composition is from about 2,500,000 cps to about 3,150,000 cps at 25° C. as measured by a Brookfield Viscometer using spindle 96 at 0.3 rpm at the 1 minute mark.

Statement 66: A method for forming a cosmetic nail coating or an artificial nail according to any one of the preceding Statements 52-65, wherein the at least one rheology modifier is selected from the group consisting of methacrylate polymers, methacrylate copolymers, styrene polymers, styrene copolymers, polyethylene, polyesters, polysulfides, polycarbonates, silicones, carboxylic esters, and any combination thereof.

Statement 67: A method for forming a cosmetic nail coating or an artificial nail according to any one of the preceding Statements 52-65, wherein the at least one rheology modifier is a carboxylic ester.

Statement 68: A method for forming a cosmetic nail coating or an artificial nail according to any one of the preceding Statements 52-65, wherein the at least one rheology modifier is a hydrogenated castor oil powder.

Statement 69: A method for forming a cosmetic nail coating or an artificial nail according to Statement 68, wherein the hydrogenated castor oil powder comprises an average particle size of from about 5 μm to about 10 μm.

Statement 70: A method for forming a cosmetic nail coating or an artificial nail according to any one of the preceding Statements 52-69, wherein the composition comprises from about 20 wt % to about 35 wt % of the at least one rheology modifier.

Statement 71: A method for forming a cosmetic nail coating or an artificial nail according to any one of the preceding Statements 52-70, wherein the rheology modifier comprises a polymer that is insoluble in the composition.

Statement 72: A method for forming a cosmetic nail coating or an artificial nail according to any one of the preceding Statements 52-71, wherein the rheology modifier comprises a polymer that forms a suspension of the polymer in the composition.

Statement 73: A method for forming a cosmetic nail coating or an artificial nail according to any one of the preceding Statements 52-72, wherein the at least one rheology modifier comprises a mean particle size of less than about 100 micrometers.

Statement 74: A method for forming a cosmetic nail coating or an artificial nail according to any one of the preceding Statements 52-72, wherein the at least one rheology modifier comprises a mean particle size of less than about 50 micrometers.

Statement 75: A method for forming a cosmetic nail coating or an artificial nail according to any one of the preceding Statements 52-72, wherein the at least one rheology modifier comprises a mean particle size of less than about 10 micrometers.

Statement 76: A method for forming a cosmetic nail coating or an artificial nail according to any one of the preceding Statements 52-72, wherein the at least one rheology modifier comprises a mean particle size of less than about 5 micrometers.

Statement 77: A method for forming a cosmetic nail coating or an artificial nail according to any one of the preceding Statements 52-76, wherein the rheology modifier is at least in part present in the composition as insoluble microspheres.

Statement 78: A method for forming a cosmetic nail coating or an artificial nail according to any one of the preceding Statements 53-77, wherein upon curing the composition by exposure to ultraviolet light, the one or more binding agents are trapped in crosslinks.

Statement 79: A method for forming a cosmetic nail coating or an artificial nail according to any one of the preceding Statements 53-78, wherein the one or more binding agents are inert and do not chemically react to form crosslinks prior to or after curing of the composition by exposure to ultraviolet light.

Statement 80: A method for forming a cosmetic nail coating or an artificial nail according to any one of the preceding Statements 52-79, wherein the composition excludes reactive oligomers and reactive polymers.

Statement 81: A method for forming a cosmetic nail coating or an artificial nail according to any one of the preceding Statements 52-80, wherein the composition only includes inert polymers.

Statement 82: A method for forming a cosmetic nail coating or an artificial nail according to any one of the preceding Statements 52-81, wherein the one or more (meth)acrylate monomers comprises a hydroxyl-containing (meth)acrylate monomer.

Statement 83: A method for forming a cosmetic nail coating or an artificial nail according to any one of the preceding Statements 52-82, wherein the one or more (meth)acrylate monomers comprises a methacrylate selected from the group consisting of hydroxypropyl methacrylate, hydroxyethyl methacrylate, ethyl methacrylate, propyl methacrylate, butyl methacrylate, isobutyl methacrylate, sec-butyl methacrylate, tetrahydrofurfuryl methacrylate, caprolactone methacrylate, methacroyloxyethyl maleate, 2-hydroxyethyl methacrylate/succinate, phthalic acid monoethyl methacrylate, and any mixture thereof.

Statement 84: A method for forming a cosmetic nail coating or an artificial nail according to any one of the preceding Statements 52-83, wherein the composition further comprises (e) one or more crosslinkers.

Statement 85: A method for forming a cosmetic nail coating or an artificial nail according to Statement 85, wherein at least one of the one or more crosslinkers is selected from the group consisting of tri(meth)acrylate, tetra(meth)acrylate, penta(meth)acrylate, and any mixture thereof.

Statement 86: A method for forming a cosmetic nail coating or an artificial nail according to Statement 85, wherein at least one of the one or more crosslinkers is an alkoxylated crosslinker having the formula (CH₂═CMe-C(O)—O-(AO)ₓ—CH₂—)₃C—R wherein R is a C₁ to C₆ group with an ‘ene’ moiety, AO is an alkoxy group selected from the group consisting of ethylene oxide, —CH₂—CH₂—O—, propylene oxide, —CH(CH₃)—CH₂—O—, —CH₂—CH₂—CH₂—O—, butylene oxide, and —CH(Et)-CH₂—O—; and wherein for each CH₂═CMe-C(O)—O-(AO)ₓ—CH₂— group x is independently 0, 1, 2, or 3.

Statement 87: A method for forming a cosmetic nail coating or an artificial nail according to Statement 85, wherein at least one of the one or more crosslinkers is an ethoxylated crosslinker having the formula (CH₂═CMe-C(O)—O—(CH₂—CH₂—O)ₓ—CH₂)₃C—C₂H₅; wherein for each CH₂═CMe-C(O)—O—(CH₂—CH₂—O)ₓ—CH₂— group x is independently 0, 1, 2, or 3.

Statement 88: A method for forming a cosmetic nail coating or an artificial nail according to Statement 85, wherein at least one of the one or more crosslinkers is selected from the group consisting of trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate, and ethoxylated isocyanuric acid tri(meth)acrylates.

Statement 89: A method for forming a cosmetic nail coating or an artificial nail according to Statement 85, wherein the one or more crosslinkers comprises tri(meth)acrylate.

Statement 90: A method for forming a cosmetic nail coating or an artificial nail according to any one of the preceding Statements 52-89, wherein at least one of the one or more photoinitiators is selected from the group consisting of 1-hydroxy-cyclohexylphenylketone, benzophenone, 2-benzyl-2-(dimethylamino)-1-(4-(4-morpholinyl)phenyl)-1-butanone, 2,2-dimethoxy-2-phenyl acetophenone, 2-methyl-1-(4-methylthio)phenyl-2-(4-morpholinyl)-1-propanone, 2,4,6-trimethylbenzoyldiphenyl-phosphine oxide, bis(2,4,6-trimethyl benzoyl)phenyl phosphine oxide, diphenyl-(2,4,6-trimethylbenzoyl) phosphine oxide, bis(2,6-dimethoxybenzoyl-2,4,4-trimethyl pentyl)phosphine oxide, 2-hydroxy-2-methyl-1-phenyl-1-propanone, phenyl bis(2,4,6-trimethylbenzoyl) phosphine oxide, benzyl-dimethylketal, isopropylthioxanthone, bis(η⁵-2,4-cyclopentadien-1-yl)bis[2,6-difluoro-3-(1H-pyrrol-1-yl)phenyl]titanium), α,α-dimethoxy α-phenyl acetophenone, ethyl (2,4,6-trimethyl benzoyl) phenyl phosphinate, phenyl (2,4,6-trimethyl benzoyl) phenyl phosphinate, methyl benzoyl formate, and any mixture thereof.

Statement 91: A method for forming a cosmetic nail coating or an artificial nail according to any one of the preceding Statements 52-90, wherein the composition comprises from about 0.5 wt % to about 5.0 wt % of the one or more photoinitiators.

Statement 92: A method for forming a cosmetic nail coating or an artificial nail according to any one of the preceding Statements 52-91, wherein the composition further comprises a colorant, a special effects pigment, or a combination thereof.

Statement 93: A method for forming a cosmetic nail coating or an artificial nail according to Statement 92, wherein the colorant is selected from the group consisting of ultramarine, manganese violet, zinc oxide, FD&C Blue No. 1, D&C Blue No. 4, Iron Blue, D&C Violet No. 2, and any mixture thereof.

Statement 94: A method for forming a cosmetic nail coating or an artificial nail according to any one of the preceding Statements 52-93, wherein the rheology modifier is selected in an amount such that a 0.5 gram sample of the composition exhibits a temperature increase of less than about 20° C. during the exposure to UV light.

Statement 95: A method for forming a cosmetic nail coating or an artificial nail according to any one of the preceding Statements 52-94, wherein the composition is stable in a dark container at 49° C. for four months or at 65° C. for 2 weeks.

Statement 96: A method for forming a cosmetic nail coating or an artificial nail according to any one of the preceding Statements 52-95, wherein the composition comprises from about 35 wt % to about 75 wt % of the one or more (meth)acrylate monomers.

Statement 97: A method for forming a cosmetic nail coating or an artificial nail according to any one of the preceding Statements 52-96, wherein the composition is a liquid or gel capable of forming an artificial nail or cosmetic nail coating in one application and without being mixed with any additional components.

Statement 98: A method of forming a cosmetic nail coating comprising: (a) placing the composition of any one of the preceding Statements 1-46 onto a nail; (b) contacting the composition with a patting solution selected from the group consisting of an acrylic nail monomer, solvent, oil, photoinitiator, and any mixture thereof; and (c) exposing the mixture to UV light.

Statement 99: A method of forming a cosmetic nail coating, the method comprising: (a) contacting the composition of any one of the preceding Statements 1-46 with a patting liquid selected from the group consisting of an acrylic nail monomer, solvent, oil, slip agent, photoinitiator, and any mixture thereof, to form a blend; (b) placing the blend onto a nail; and (c) exposing the blend to UV light.

We claim:

1. A photopolymerizable composition comprising:
(a) from about 15 wt % to about 45 wt % of at least one rheology modifier;
(b) from about 25 wt % to about 75 wt % of one or more (meth)acrylate monomers;
(c) one or more photoinitiators; and
(d) from about 15 wt % to about 45 wt % of one or more binding agents.

2. The composition according to claim 1, wherein the one or more binding agents comprises one or more selected from the group consisting essentially of polyisobutene, ozokerite wax, ceresin, and a copolymer of acrylamide and sodium acryloyldimethyltaurate.

3. The composition according to claim 1, wherein the one or more binding agents comprises acrylamide, sodium acryloyldimethyl taurate copolymer, isohexadecane, polysorbate 80, and any combination thereof.

4. The composition according to claim 1, wherein the one or more binding agents is not a wax.

5. The composition according to claim 1, wherein the at least one rheology modifier is selected in an amount such that the viscosity of the composition is greater than 400,000 centipoise (cps) at 25° C. as measured by a cone and plate rheometer using 40 mm 1° steel cone with a truncation gap of 31 microns at a 10 s⁻¹ shear rate on a 500 mg sample.

6. The composition according to claim 1, wherein the at least one rheology modifier is selected from the group consisting of methacrylate polymers, methacrylate copolymers, styrene polymers, styrene copolymers, polyethylene, polyesters, polysulfides, polycarbonates, silicones, carboxylic esters, and any combination thereof.

7. The composition according to claim 1, wherein the rheology modifier comprises a polymer that is insoluble in the composition.

8. The composition according to claim 1, wherein the rheology modifier is at least in part present in the composition as insoluble microspheres.

9. The composition according to claim 1, wherein the one or more binding agents are inert and do not chemically react to form crosslinks prior to or after curing of the composition by exposure to ultraviolet light.

10. The composition according to claim 1, wherein the composition excludes reactive oligomers and reactive polymers.

11. The composition according to claim 1, wherein the composition only includes inert polymers.

12. The composition according to claim 1, wherein the one or more (meth)acrylate monomers comprises a methacrylate selected from the group consisting of hydroxypropyl methacrylate, hydroxyethyl methacrylate, ethyl methacrylate, propyl methacrylate, butyl methacrylate, isobutyl methacrylate, sec-butyl methacrylate, tetrahydrofurfuryl methacrylate, caprolactone methacrylate, methacroyloxyethyl maleate, 2-hydroxyethyl methacrylate/succinate, phthalic acid monoethyl methacrylate, and any mixture thereof.

13. The composition according to claim 1, further comprising (e) one or more crosslinkers.

14. The composition according to claim 1, wherein the rheology modifier is selected in an amount such that a 0.5 gram sample of the composition exhibits a temperature increase of less than about 20° C. during the exposure to UV light.

15. The composition according to claim 1, wherein the composition is stable in a dark container at 49° C. for four months or at 65° C. for 2 weeks.

16. The composition according to claim 1, wherein the composition is a liquid or gel capable of forming an artificial nail or cosmetic nail coating in one application and without being mixed with any additional components.

17. An artificial nail or cosmetic nail coating kit comprising: at least one first container containing an artificial nail sculpting composition according to claim 1; and at least one second container containing a patting solution for contacting with the artificial nail sculpting composition to aid the spreading and shaping of the artificial nail sculpting composition on a nail.

18. A method for forming a cosmetic nail coating or an artificial nail, the method comprising:
applying a composition onto a nail in one application step, the composition comprising:
(a) from about 15 wt % to about 45 wt % of at least one rheology modifier;
(b) from about 25 wt % to about 75 wt % of one or more (meth)acrylate monomers;
(c) one or more photoinitiators; and
(d) from about 15 wt % to about 45 wt % of one or more binding agents.
exposing the composition to UV light;
wherein the composition is in the form of a liquid or gel and is not mixed with any additional components prior to the applying of the composition onto the nail.

19. The method according to claim 18, wherein the one or more binding agents comprises one or more selected from the group consisting essentially of one or more selected from the group consisting essentially of polyisobutene, ozokerite wax, ceresin, and a copolymer of acrylamide and sodium acryloyldimethyltaurate.

20. The method according to claim 18, wherein the composition excludes reactive oligomers and reactive polymers.
